(19) United States
(12) Patent Application Publication
(10) Pub. No.: US 2016/0296134 A1
(43) Pub. Date: Oct. 13, 2016

(54) ELECTRONIC DEVICE AND INFORMATION PROCESSING METHOD

(71) Applicants: Beijing Lenovo Software Ltd., Beijing (CN); Lenovo (Beijing) Limited, Beijing (CN)

(72) Inventors: Bin Li, Beijing (CN); Ka'er Huang, Beijing (CN)

(21) Appl. No.: 14/847,719

(22) Filed: Sep. 8, 2015

(30) Foreign Application Priority Data
Apr. 7, 2015 (CN) 201510161136.5

(51) Int. Cl. A61B 5/053; A61B 5/00

(52) U.S. Cl. CPC A61B 5/0531; A61B 5/681; A61B 5/6803; A61B 5/7278

(57) ABSTRACT

The present disclosure discloses an electronic device and an information processing method. The electronic device comprises a detection unit configured to detect biological parameters of a user wearing the electronic device. Specifically, in an embodiment of the present disclosure, the electronic device is a wearable portable electronic device, and a first detection contact point and a second detection contact point comprised by the detection unit for detecting the biological parameters of the user are two electrodes, respectively. Thus, the electronic device which is worn on the body of a user can rapidly detect body impedance of the user through signal interaction between the two electrodes, so as to reduce the cost of detection of the body impedance and also improve the convenience in detection of the body impedance.

### CROSS-REFERENCE TO RELATED APPLICATION(S)

This application claims priority to the Chinese Patent Application No. 201510161136.5, filed on Apr. 7, 2015, which application is incorporated herein by reference in its entirety.

### TECHNICAL FIELD

The present disclosure relates to the field of electronic technology, and in particular, to an electronic device and an information processing method.

### BACKGROUND

With the development of electronic technology, there are increasingly various portable terminal devices, for example, a mobile phone, a tablet computer or the like.

Currently, some electronic devices are capable of detecting a part of biological parameters of a user, for example, fingerprint information, body temperature or the like, and then the electronic devices may control some functions thereof according to the detected biological parameters.

However, there is still a need for professional devices to detect some kind of biological parameters, such as biological parameters related to body impendence, which results in a high cost of detection and inconvenience for user.

### SUMMARY

Embodiments of the present disclosure provide an electronic device and an information processing method.

An electronic device comprises: a detection unit configured to detect biological parameters of a user wearing the electronic device, wherein the detection unit comprises at least a first detection contact point and a second detection contact point, and the detection unit is operative to detect the biological parameters through signal interaction between the first detection contact point and the second detection contact point.

Alternatively, the electronic device further comprises: a main body portion; and a maintaining portion connected to the main body portion and configured to maintain a relative positional relationship with respect to at least a part of the body of the user, wherein the detection unit is arranged in the main body portion and/or the maintaining portion.

Alternatively, the maintaining portion has at least a fixed state, and the maintaining portion is provided as at least a part of an annular space or at least a part of an approximate annular space which satisfies a first preset condition.

Alternatively, an outer surface of the annular space comprises an inner annular surface and an outer annular surface, and the inner annular surface has a diameter less than that of the outer annular surface, the first detection contact point and the second detection contact point of the detection unit are arranged on the inner annular surface, and a distance between the first detection contact point and the second detection contact point is greater than or equal to a preset threshold, so that the first detection contact point and the second detection contact point are in contact with the user when the relative positional relationship between the electronic device and the user is maintained.

Alternatively, the electronic device comprises a first outer surface and a second outer surface, and a distance between the first outer surface and the user is less than a distance between the second outer surface and the user when the maintaining portion maintains a fixed relationship with respect to the user; and wherein the first detection contact point of the detection unit is arranged on the first outer surface and the second detection contact point is arranged on the second outer surface, so that the first detection contact point is in contact with the user and the second detection contact point is provided as a detection point to be contacted.

Alternatively, the electronic device further comprises: a processor configured to process a data signal; a signal generator connected to the processor and the first detection contact point, the signal generator is configured to generate an excitation signal according to a trigger signal transmitted by the processor, and transmit the excitation signal to the first detection contact point, so that the first detection contact point is operative to transmit the excitation signal to the user; and a signal receiver connected to the processor and the second detection contact point, the signal receiver is configured to receive a detection signal detected by the second detection contact point, and transmit the detection signal to the processor, so that the processor is operative to obtain the biological parameters of the user.

Alternatively, the processor is further configured to control the signal generator to generate the excitation signal, to receive the detection signal received by the signal receiver, and to obtain the biological parameters according to the excitation signal and the detection signal.

Alternatively, the first detection contact point is a first electrode and the second detection contact point is a second electrode.

An information processing method comprises: detecting, by a detection unit of an electronic device, biological parameters of a user wearing the electronic device, wherein the detection unit comprises at least a first detection contact point and a second detection contact point, and the detection unit is operative to detect the biological parameters of the user through signal interaction between the first detection contact point and the second detection contact point.

Alternatively, the detecting, by a detection unit of an electronic device, biological parameters of a user wearing the electronic device comprises: transmitting, by the first detection contact point, an excitation signal from a signal generator in the electronic device to the user; obtaining, by the second detection contact point, a detection signal from at least a part of the body of the user; and obtaining the biological parameters of the user according to the excitation signal and the detection signal.

The embodiments of the present disclosure provide an electronic device, which comprises: a detection unit configured to detect biological parameters of a user wearing the electronic device, wherein the detection unit at least comprises a first detection contact point and a second detection contact point, and the detection unit can detect the biological parameters of the user through signal interaction between the first detection contact point and the second detection contact point. Specifically, in the embodiments of the present disclosure, the electronic device can rapidly detect body impedance of the user through signal interaction between the first detection contact point and the second detection contact point in the electronic device, so as to reduce the cost of detection of the body impedance and also improve the convenience in detection of the body impedance.

### BRIEF DESCRIPTION OF THE DRAWINGS

FIG. 1 is a first structural diagram of an electronic device according to an embodiment of the present disclosure;

FIG. 2 is a second structural diagram of an electronic device according to an embodiment of the present disclosure;

FIG. 3A is a structural diagram of a maintaining portion according to an embodiment of the present disclosure;

FIG. 3B is another structural diagram of a maintaining portion according to an embodiment of the present disclosure; and

FIG. 4 is a third structural diagram of an electronic device according to an embodiment of the present disclosure.

### DETAILED DESCRIPTION

The embodiments of the present disclosure provide an electronic device, which comprises: a detection unit configured to detect biological parameters of a user wearing the electronic device, wherein the detection unit at least comprises a first detection contact point and a second detection contact point, and the detection unit can detect the biological parameters of the user through signal interaction between the first detection contact point and the second detection contact point. Specifically, in the embodiments of the present disclosure, the electronic device can rapidly detect body impedance of the user through signal interaction between the first detection contact point and the second detection contact point in the electronic device, so as to reduce the cost of detection of the body impedance and also improve the convenience in detection of the body impedance.

The technical solutions of the present disclosure will be described in detail below in conjunction with accompanying drawings and specific embodiments. It should be understood that the embodiments of the present disclosure and the specific technical features in the embodiments are used to explain the technical solutions of the present disclosure, instead of limiting the technical solutions of the present disclosure. Without a conflict, the embodiments of the present disclosure and the specific technical features in the embodiments can be combined with each other.

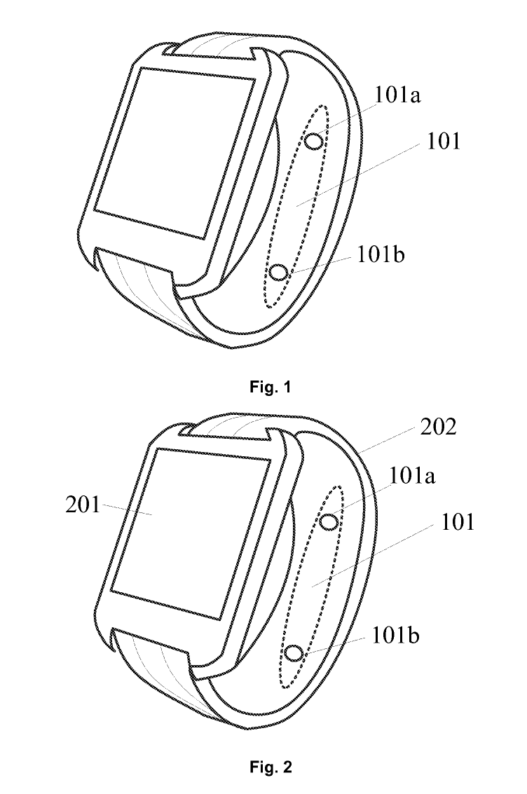

As shown in FIG. 1, illustrated is a structural diagram of an electronic device according to an embodiment of the present disclosure. The electronic device comprises: a detection unit 101 configured to detect biological parameters of a user wearing the electronic device, wherein the detection unit at least comprises a first detection contact point 101a and a second detection contact point 101b, and the detection unit 101 may detect the biological parameters of the user through signal interaction between the first detection contact point 101a and the second detection contact point 101b.

Firstly, in the embodiment of the present disclosure, the electronic device comprises a detection unit 101. The detection unit 101 is arranged in the electronic device which may be worn on a body of a user, so that the detection unit 101 may directly detect biological parameters of the user.

Alternatively, in the embodiment of the present disclosure, in order to enable the detection unit 101 to collect the biological parameters of the user, the detection unit 101 comprises a first detection contact point 101a and a second detection contact point 101b. The first detection contact point 101a and the second detection contact point 101b have respective detection functions. For example, both the first detection contact point 101a and the second detection contact point 101b may detect an electrical pulse signal or the like of the body of the user.

Further, in the embodiment of the present disclosure, in order to enable the electronic device to transmit a signal through the first detection contact point 101a and detect the biological parameters of the user through the second detection contact point 101b, the first detection contact point 101a and the second detection contact point 101b may be used cooperatively. For example, the first detection contact point 101a is used to transmit an excitation signal, and then the second detection contact point 101b is used to receive a detection signal, so that the first detection contact point 101a and the second detection contact point 101b are used cooperatively to obtain the biological parameters of the user wearing the electronic device. It should be noted here that in the embodiment of the present disclosure, in order to ensure that the detection unit 101 can detect the biological parameters of the user, the detection unit 101 at least comprises two detection contact points, and the two detection contact points need to be used cooperatively. That is to say, it is impossible to detect the biological parameters of the user by merely using a single detection contact point.

Further, in the embodiment of the present disclosure, the electronic device may be a wearable device, for example, smart glasses, smart watch, smart helmet or the like. In order to ensure that the electronic device can be conveniently worn on a certain part of the body of the user, the electronic device comprises a structure illustrated in FIG. 2, which may comprise: a main body portion 201; and a maintaining portion 202 connected to the main body portion 201 and configured to maintain a relative positional relationship with respect to at least a part of the body of the user.

The detection unit is arranged in the main body portion 201 and/or the maintaining portion 202.

Further, the maintaining portion 202 is connected to the main body portion 201, and is configured to maintain a relative positional relationship with respect to at least a part of the body of the user. The maintaining portion 202 has at least a fixed state, and may be used as at least a part of an annular space or an annulus-like space which satisfies a first preset condition. The annular space or the annulus-like space may surround a periphery of a cylindrical body which satisfies a second preset condition.

In brief, the electronic device is comprised of a main body portion 201 and a maintaining portion 202. The electronic device may be fixed to a certain part of an operation body through the maintaining portion 202. For example, the electronic device may be a smart wearable device such as a smart watch worn on the wrist, smart glasses worn on the head, a smart head ring worn on the head or the like.

Alternatively, in the embodiment of the present disclosure, the main body portion 201 and/or the maintaining portion 202 of the electronic device may have a battery, a chip, a memory card or the like arranged therein, which may be set by an ordinary skilled in the art according to practical requirements, and will not be limited in the present disclosure.

The maintaining portion 202 may be a fixing member for fixing the electronic device, for example, a watchband of a watch, a head-mounted rack or the like.

When the maintaining portion 202 is in a fixed state, the maintaining portion 202 may be used as at least a part of an annular space or an annulus-like space which satisfies a first preset condition. The annular space or the annulus-like space may surround a periphery of a cylindrical body which satisfies a second preset condition.

Specifically, the maintaining portion 202 may be implemented in many manners, two of which will be described below. In a specific implementation process, the manners for implementing the maintaining portion 202 include but are not limited to the following two manners.

In a first manner, the maintaining portion 202 has at least a fixed state, and may be used as at least a part of an annular space or an annulus-like space which satisfies a first preset condition. The annular space or the annulus-like space may surround a periphery of an operation body which satisfies a second preset condition.

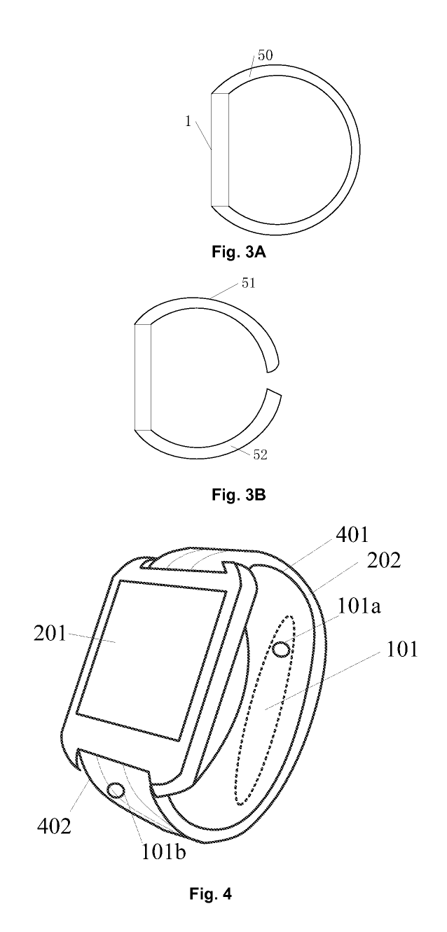

Specifically, when the maintaining portion 202 is merely a single part of the annular space or the annulus-like space, the maintaining portion 202 may form an annular space together with the main body portion where a display 1 is located. As shown in FIG. 3A, a digit 50 represents the maintaining portion 202.

With reference to FIG. 3B, when the maintaining portion 202 comprises a first fixing part represented by a digital 51 and a second fixing part represented by a digit 52, a first end of the first fixing part is connected to a first side of the display 1, and a third end of the second fixing part is connected to a second side of the display 1. The first side and the second side are two sides of the display 1 which are opposite to each other.

In this case, if the maintaining portion 202 is of a hard material, and a second end of the first fixing part and a fourth end of the second fixing part are in a non-connected state, the maintaining portion 202 is in a fixed state, and the first fixing part and the second fixing part are two parts of an annulus-like space. The annulus-like space satisfies a first preset condition, i.e., a distance between the second end of the first fixing part and the fourth end of the second fixing part is less than a diameter of a part of the body, for example, the wrist, on which the electronic device is worn. In this case, the wrist is treated as a cylindrical operation body. The cylindrical operation body satisfies a second preset condition, i.e., a diameter of a part of the cylindrical operation body is greater than a diameter of the annular space. For example, if a perimeter of the wrist is 10 cm, and an inner diameter of the annular space is 12 cm, a perimeter of a part of the wearer which connects the wrist to the palm should be at least greater than 12 cm, so as to avoid the worn electronic device from sliding down.

In a second manner, the maintaining portion 202 has at least a fixed state, and may be used as at least a part of an annular space or an annulus-like space which satisfies a first preset condition. The annular space or the annulus-like space may surround a periphery of a sphere-like body which satisfies a third preset condition.

The same content of the second manner as that of the first manner will not be described here. The second manner differs from the first manner in that when the electronic device is worn on a certain part of the body of the user, for example, the head, the head may be treated as a sphere-like body. The sphere-like body satisfies a third preset condition, i.e., a diameter of the sphere-like body is greater than a diameter of the annular space or the annulus-like space.

In a specific implementation, the maintaining portion 202 is required to maintain a relative positional relationship between the electronic device and a first part of the body of the user. In general, attribute parameters of the maintaining portion 202 should match physical parameters of the user. For example, if it needs to fix the electronic device to the wrist of the user, an effective perimeter of the maintaining portion 202 which surrounds the wrist when the maintaining portion 202 maintains the relative positional relationship should be greater than or equal to a perimeter of a periphery of the wrist. For example, if the perimeter of the wrist is typically between 10 cm and 15 cm, the effective perimeter of the maintaining portion 202 should be at least equal to 15 cm. Alternatively, for example, if it needs to fix the electronic device to the head of the user, since a perimeter of a forehead of an adult is between 54 cm and 58 cm, the perimeter of the maintaining portion 202 may be 59 cm or the like, which can be set by an ordinary skilled in the art according to practical requirements, and will not be limited in the present disclosure.

Further, in the embodiment of the present disclosure, an outer surface of the annular space of the maintaining portion 202 comprises an inner annular surface and an outer annular surface. A diameter of the inner annular surface is less than a diameter of the outer annular surface. The first detection contact point 101a and the second detection contact point 101b of the detection unit 101 are arranged in the inner annular surface, and a distance between the first detection contact point 101a and the second detection contact point 101b is greater than or equal to a preset threshold, so that the first detection contact point 101a and the second detection contact point 101b can be in contact with the user when a relative positional relationship between the electronic device and the user is maintained.

Specifically, in FIG. 2, the electronic device is specifically a smart watch and the maintaining portion 202 is specifically a watchband part of the smart watch. The watchband part has an inner annular surface and an outer annular surface. Obviously, a diameter of the inner annular surface of the watchband part is less than a diameter of the outer annular surface of the watchband part. When the user wears the smart watch, the inner annular surface of the watchband part is in contact with the wrist of the user, and the outer annular surface is away from the user. The first detection contact point 101a is arranged in the inner annular surface of the watchband part of the smart watch. Alternatively, the second detection contact point 101b is also arranged in the inner annular surface of the watchband part.

Alternatively, a distance between the first detection contact point 101a and the second detection contact point 101b is greater than or equal to a preset threshold. For example, the distance between the first detection contact point 101a and the second detection contact point 101b may be set as 5 cm. The distance between the first detection contact point 101a and the second detection contact point 101b may be increased to reduce the mutual influences between the two detection contact points. In addition, after the distance between the first detection contact point 101a and the second detection contact point 101b is increased, an excitation signal transmitted by the first detection contact point 101a may be transmitted for a long distance in the body of the user, so that a signal received by the second detection contact point 101b is a signal obtained after the excitation signal passes through essentially the whole body impedance. Therefore, the distance between the first detection contact point 101a and the second detection contact point 101b may be increased to ensure that the finally obtained body impedance is close to the actual body impedance, so as to improve the accuracy of the finally detected biological parameters.

Alternatively, in the embodiment of the present disclosure, the electronic device may further comprise a first outer surface and a second outer surface. When the maintaining portion 202 maintains a fixed relationship with respect to the user, a distance between the first outer surface and the user is less than a distance between the second outer surface and the user. The first detection contact point 101a of the detection unit is arranged in the first outer surface, and the second detection contact point 101b of the detection unit is arranged in the second outer surface, so that the first detection contact point is in contact with the user, and the second detection contact point is used as a detection contact point to be contacted. The first detection contact point 101a is arranged in the first outer surface, and the second detection contact point 101b is arranged in the second outer surface, so that the excitation signal transmitted by the first detection contact point 101a may be transmitted for a long distance in the body of the user. In this way, a signal received by the second detection contact point 101b is a signal obtained after the excitation signal passes through essentially the whole body impedance. Therefore, the distance between the first detection contact point 101a and the second detection contact point 101b may be increased to ensure that the finally obtained body impedance is closer to the actual body impedance, so as to improve the accuracy of detection of the body impedance.

Specifically, as shown in FIG. 4, the electronic device is a smart watch illustrated in FIG. 4. An outer surface of the smart watch comprises a first outer surface 401 and a second outer surface 402. After a user wears the smart watch, the first outer surface 401 of the smart watch is closer to the wrist of the user, and the second outer surface 402 is away from the wrist of the user. A first detection contact point is arranged in the first outer surface 401 of the smart watch. When the user wears the smart watch, the first detection contact point 101a in the first outer surface 401 is in contact with the wrist of the user. Alternatively, a second detection contact point 101b is arranged in the second outer surface 402 of the smart watch. The second detection contact point 101b is used as a detection contact point to be contacted. That is, when the user needs to detect his/her body impedance, the user may enable a certain part of the body to be in contact with the second detection contact point 101b, for example, the user enables his/her finger to be in contact with the second detection contact point 101b, or enables his/her leg to be in contact with the second detection contact point 101b. In this way, the first detection contact point 101a, the body of the user, and the second detection contact point 101b may form a complete loop. Thus, the signal interaction between the first detection contact point 101a and the second detection contact point 101b may enable the signal to be transmitted for a long distance in the body of the user, so as to enable the smart watch to more accurately detect the body impedance of the user.

Alternatively, the first contact point 101a is disposed at a first position on the first outer surface 401, and the second contact point 101b is disposed at a second position on the second outer surface 402 corresponding to the first position. In one embodiment, the first and second positions are opposite sides of a position at the maintaining portion of the smart watch. When a user wants to detect biological parameter, he/she wears the smart watch on one of the wrists, and the first contact point 101a is brought to be in contact with the wrist. When the user puts another hand or any other part of his/her body on the second outer surface and presses on the second contact point 101b, the first contact point 101a disposed on the first outer surface 401 will be fitting to the user's wrist more closely under the pressure at the second contact point 101b, thereby accuracy of detection is further improved.

Further, in the embodiment of the present disclosure, both the first detection contact point 101a and the second detection contact point 101b of the electronic device may be arranged in the second outer surface. During detection, the first detection contact point 101a is in contact with a certain part of the body of the user, and the second detection contact point 101b is in contact with another part of the body of the user, so that the first detection contact point 101a, the body of the user, and the second detection contact point 101b may form a loop. Thus, after an excitation signal transmitted by the first detection contact point 101a passes through the body of the user, the signal is received by the second detection contact point 101b. The signal detected by the second detection contact point 101b may be used to obtain the body impedance of the user.

Alternatively, in the embodiment of the present disclosure, the first detection contact point 101a and the second detection contact point 101b may be arranged radially in opposite positions of the second outer surface. For example, for the smart watch illustrated in FIG. 3B, the first detection contact point 101a may be arranged in an upper outer surface of the watchband of the smart watch, and the second detection contact point 101b may be arranged in a lower outer surface of the watchband of the smart watch. Thus, the user may conveniently be in contact with the two detection contact points. For example, the user may be in contact with the detection contact point in the upper outer surface of the watchband with a hand, and then may be in contact with the detection contact point in the lower outer surface of the watchband with a leg. Thus, the first detection contact point 101a, the body of the user, and the second detection contact point 101b form a detection loop, and an excitation signal transmitted by the first detection contact point 101a may also be transmitted for a long distance in the body of the user, so that body impedance of the user may be more accurately calculated according to a signal detected by the second detection contact point 101b.

It should be illustrated that in the embodiment of the present disclosure, two or more detection contact points are required to be used in the detection unit 101, for example, the first detection contact point 101a and the second detection contact point 101b as described above, so that an excitation signal is transmitted by the first detection contact point to the body of the user, and then the detection signal from the body of the user is detected by the second detection contact point, so as to finally obtain the body impedance of the user.

Alternatively, in addition to using two detection contact points to detect the body impedance as described above, three, four, or more than four detection contact points may also be used to detect the body impedance. For example, when three detection contact points are arranged in the electronic device, still by taking the electronic device comprising a first outer surface 401 and a second outer surface 402 as an example, two of the three detection contact points may be arranged in the first outer surface 401 of the electronic device, and another detection contact point may be arranged in the second outer surface 402. When the user wears the electronic device, two detection contact points in the first outer surface are in contact with the body of the user, and another detection contact point is used as a detection contact point to be contacted.

For example, the electronic device is a smart watch. Two detection contact points are arranged in an inner surface of the watchband part of the smart watch. That is, two detection contact points are arranged in a surface which is closer to the wrist of the user when the user wears the smart watch. Alternatively, a distance between the two detection contact points is greater than or equal to a preset threshold, for example, 5 cm. Then, a third detection contact point is arranged in an outer surface, and is used as a detection contact point to be contacted. As the detection distance between the detection contact points is proportional to the accuracy of detection of the impedance, when a user needs to rapidly detect the body impedance, two detection contact points in the inner surface of the smart watch may be used to detect the body impedance, and if the user needs to more accurately detect his/her body impedance, the user may use one detection contact point in the inner surface and one detection contact point in the outer surface to achieve high accuracy of detection of the body impedance.

Alternatively, in the embodiment of the present disclosure, four or more detection contact points may be used. For example, two detection contact points may be arranged in the inner surface of the smart watch, and then two detection contact points may also be arranged in the outer surface of the smart watch. The two detection contact points in the inner surface of the smart watch are used to detect a detection signal from the body of the user, and the two detection contact points in the outer surface of the smart watch are used to transmit an excitation signal to the user. Alternatively, only the two detection contact points in the inner surface of the smart watch may be used to detect the body impedance. The four detection contact points may also be used to more accurately detect the body impedance.

Alternatively, in the embodiment of the present disclosure, 5 or more detection contact points may also be used to detect the body impedance, which will not be described here.

Alternatively, in the embodiment of the present disclosure, three detection contact points are used. The manner of detecting the body impedance by using three detection contact points has been described in the above embodiment in detail, and will not be described here.

In order to enable the electronic device to obtain the biological parameters of the user through the first detection contact point 101a and the second detection contact point 101b, i.e., the body impedance of the user, the electronic device further comprises: a processor (not shown) configured to process a data signal; a signal generator (not shown) connected to the processor and the first detection contact point 101a, and configured to generate an excitation signal according to a trigger signal transmitted by the processor, and transmit the excitation signal to the first detection contact point 101a, so that the first detection contact point 101a transmits the excitation signal to the user; and a signal receiver (not shown) connected to the processor and the second detection contact point 101b, and configured to receive a detection signal detected by the second detection contact point 101b, and transmit the detection signal to the processor, so that the processor obtains the biological parameters of the user.

For example, the electronic device is a smart watch, which has a processor, a signal generator, and a signal receiver arranged therein. The processor is connected to the signal generator and the signal receiver respectively. The signal generator is connected to a first detection contact point 101a, and the signal receiver is connected to a second detection contact point 101b. When the smart watch is used to detect body impedance, the processor in the smart watch transmits a trigger signal to the signal generator, and after receiving the trigger signal, the signal generator generates an excitation signal, and transmits the excitation signal to the body of the user through the first detection contact point 101a.

In this case, the second detection contact point 101b in the smart watch obtains the detection signal from the body of the user in real time. The detection signal is transmitted to the signal receiver through the second detection contact point 101b, and the signal receiver finally transmits the received detection signal to the processor. The processor receives the detection signal transmitted by the signal receiver and the excitation signal transmitted by the signal generator, and substitutes them into a predetermined operational formula. Then, a calculated result is used by the processor as the biological parameters of the user, i.e., the body impedance of the user. Obliviously, this can not only reduce the cost of detection of the body impedance, but also can more accurately and conveniently detect the body impedance of the user.

It should be further illustrated here that in the embodiment of the present disclosure, the first detection contact point 101a and the second detection contact point 101b may respectively be a first electrode and a second electrode which have essentially the same shape and structure. That is, the first detection contact point 101a and the second detection contact point 101b may simply be two metallic conductors, which are primarily used to conduct signals, i.e., one metallic conductor is used to transmit a signal, and the other metallic conductor is used to receive a signal. The first detection contact point 101a and the second detection contact point 101b are used cooperatively, so that a detection unit which comprises the first detection contact point 101a and the second detection contact point 101b may detect the biological parameters of the user, i.e., the body impedance of the user. Specifically, in the embodiment of the present disclosure, the first detection contact point 101a transmits an excitation signal to the body of the user, and after the excitation signal is transmitted in the body of the user, the second detection contact point 101b may detect the detection signal from the body of the user, so that biological parameters of the user are detected through signal interaction between the first detection contact point 101a and the second detection contact point 101b.

In general, in the embodiment of the present disclosure, the electronic device comprises a detection unit 101 which comprises a first detection contact point 101a and a second detection contact point 101b. The electronic device may detect the biological parameters of the user wearing the electronic device, i.e., the body impedance of the user, through the first detection contact point 101a and the second detection contact point 101b. This can not only reduce the cost of detection of the body impedance, but also can more rapidly and conveniently detect the body impedance.

In the embodiment of the present disclosure, an information processing method corresponding to the electronic device according to the embodiment of the present disclosure is further provided. The method comprises: detecting, by a detection unit in the electronic device, biological parameters of a user wearing the electronic device.

The detection unit at least comprises a first detection contact point and a second detection contact point, and the detection unit may detect the biological parameters of the user through signal interaction between the first detection contact point and the second detection contact point.

Specifically, in the embodiment of the present disclosure, the electronic device is described in the above embodiment, which comprises a first detection contact point and a second detection contact point. The electronic device transmits an excitation signal from the signal generator in the electronic device to the user through the first detection contact point. That is, when the user needs to detect his/her body impedance, the first detection contact point in the electronic device transmits an excitation signal to the body. Then, the second detection contact point in the electronic device obtains a detection signal from at least a part of the body of the user.

When the second detection contact point detects the detection signal, the processor in the electronic device obtains the excitation signal and the detection signal, and then substitutes the excitation signal and the detection signal into a predetermined algorithm. Finally, a calculated result is used by the processor in the electronic device as the body impedance of the user. With the above method, the body impedance of the user can be conveniently and rapidly detected, the cost of detection of the body impedance can be reduced, and the efficiency in detection of the body impedance can be improved.

Alternatively, in the embodiment of the present disclosure, the electronic device may be an intelligent portable electronic device such as a smart watch, a smart wristband, or smart glasses or the like.

Although preferable embodiments of the present disclosure have been described, additional changes and modifications can be made to these embodiments by those skilled in the art upon learning the basic creative concepts. Therefore, the appended claims are intended to be construed as comprising the preferable embodiments and all changes and modifications that fall into the scope of the present disclosure.

Obviously, those skilled in the art can make various modifications and variations to the present disclosure without departing from the spirit and scope of the present disclosure. Thus, if these modifications and variations of the present disclosure belong to the scope of the claims of the present disclosure and the equivalent technologies thereof, the present disclosure is also intended to include these modifications and variations.

I/we claim:

1. An electronic device, comprising:
a detection unit configured to detect biological parameters of a user wearing the electronic device, wherein the detection unit comprises at least a first detection contact point and a second detection contact point, and the detection unit is operative to detect the biological parameters through signal interaction between the first detection contact point and the second detection contact point.

2. The electronic device according to claim 1, further comprising:
a main body portion; and
a maintaining portion connected to the main body portion and configured to maintain a relative positional relationship with respect to at least a part of the body of the user,
wherein the detection unit is arranged in the main body portion and/or the maintaining portion.

3. The electronic device according to claim 2, wherein, the maintaining portion has at least a fixed state, and the maintaining portion is provided as at least a part of an annular space or at least a part of an approximate annular space which satisfies a first preset condition.

4. The electronic device according to claim 3, wherein:
an outer surface of the annular space comprises an inner annular surface and an outer annular surface, and the inner annular surface has a diameter less than that of the outer annular surface,
the first detection contact point and the second detection contact point of the detection unit are arranged on the inner annular surface, and a distance between the first detection contact point and the second detection contact point is greater than or equal to a preset threshold, so that the first detection contact point and the second detection contact point are in contact with the user when the relative positional relationship between the electronic device and the user is maintained.

5. The electronic device according to claim 2, wherein the electronic device comprises a first outer surface and a second outer surface, and a distance between the first outer surface and the user is less than a distance between the second outer surface and the user when the maintaining portion maintains a fixed relationship with respect to the user; and wherein the first detection contact point of the detection unit is arranged on the first outer surface and the second detection contact point is arranged on the second outer surface, so that the first detection contact point is in contact with the user and the second detection contact point is provided as a detection point to be contacted.

6. The electronic device according to claim 1, further comprising:
a processor configured to process a data signal;
a signal generator connected to the processor and the first detection contact point, the signal generator is configured to generate an excitation signal according to a trigger signal transmitted by the processor, and transmit the excitation signal to the first detection contact point, so that the first detection contact point is operative to transmit the excitation signal to the user; and
a signal receiver connected to the processor and the second detection contact point, the signal receiver is configured to receive a detection signal detected by the second detection contact point, and transmit the detection signal to the processor, so that the processor is operative to obtain the biological parameters of the user.

7. The electronic device according to claim 6, wherein the processor is further configured to control the signal generator to generate the excitation signal, to receive the detection signal received by the signal receiver, and to obtain the biological parameters according to the excitation signal and the detection signal.

8. The electronic device according to claim 1, wherein the first detection contact point is a first electrode and the second detection contact point is a second electrode.

9. An information processing method, comprising:
detecting, by a detection unit of an electronic device, biological parameters of a user wearing the electronic device, wherein the detection unit comprises at least a first detection contact point and a second detection contact point, and the detection unit is operative to detect the biological parameters of the user through signal interaction between the first detection contact point and the second detection contact point.

10. The method according to claim 9, wherein detecting, by a detection unit of an electronic device, biological parameters of a user wearing the electronic device comprises:
transmitting, by the first detection contact point, an excitation signal from a signal generator in the electronic device to the user;
obtaining, by the second detection contact point, a detection signal from at least a part of the body of the user; and
obtaining the biological parameters of the user according to the excitation signal and the detection signal.
